(19) United States
(12) Patent Application Publication
(10) Pub. No.: US 2026/0157683 A1
(43) Pub. Date: Jun. 11, 2026

(54) EVALUATION METHOD AND DEVICE FOR HEAD IMPULSE TEST

(71) Applicants: Korea University Research and Business Foundation, Seoul (KR); SEOUL NATIONAL UNIVERSITY HOSPITAL, Seoul (KR)

(72) Inventors: Sun-Uk LEE, Seoul (KR); Byung Jo KIM, Seoul (KR); Jung Bin KIM, Seoul (KR); Sung Hwan KIM, Seoul (KR); Jeongyoon CHOI, Seongnam-si, Gyeonggi-do (KR)

(73) Assignees: Korea University Research and Business Foundation, Seoul (KR); SEOUL NATIONAL UNIVERSITY HOSPITAL, Seoul (KR)

(21) Appl. No.: 18/706,040

(22) PCT Filed: Nov. 1, 2022
(86) PCT No.: PCT/KR2022/016865

§ 371 Date: May 29, 2024

(30) Foreign Application Priority Data
Nov. 1, 2021 (KR) 10-2021-0147864
Oct. 31, 2022 (KR) 10-2022-0143016

(51) Int. Cl. A61B 5/00; A61B 5/11; A61B 5/16

(52) U.S. Cl. CPC A61B 5/4064; A61B 5/0077; A61B 5/1103; A61B 5/1114; A61B 5/1128; A61B 5/163; A61B 5/4884; A61B 5/7207; A61B 5/725; A61B 5/7282

(57) ABSTRACT

An evaluation method for a head impulse test includes a step of extracting subject's eye and head movement data by a video nystagmograph; a step of detecting an eye movement that is not synchronized with a horizontal head movement by a head impulse tester; a step of obtaining corrective saccade data by removing noise from an unsynchronized eye movement signal detected in the head impulse tester by a filter unit; and a step of calculating a ratio of horizontal to vertical angular velocity of corrective saccades as a quantitative index by a control unit. The present invention is dramatically helpful in the diagnosis of central brain lesions by detecting the vertical abnormal trajectory in the central brain lesions. In addition, by calculating quantified data of the vertical abnormal trajectory

### TECHNICAL FIELD

The present invention relates to an evaluation method and a device for a head impulse test device, and more specifically, relates to an evaluation method and a device for a head impulse test in which a result of a head impulse test is able to be evaluated by receiving eye movement data from a video nystagmography and calculating a ratio of horizontal to vertical corrective saccades as a quantitative index.

### BACKGROUND

Dizziness may be broadly divided into those caused by the central or peripheral nervous system, and certain types of dizziness are known to have unique eye movement patterns.

Three semicircular canals of the human body perceive a rotational angular acceleration centered on X, Y, and Z-axes in a three-dimensional space, and the otolith organs perceive horizontal and vertical linear accelerations thereby performing the role of a gyro sensor that transmits signals thereof to the central nervous system.

These vestibular signals transmitted to the central nervous system according to changes in head position trigger the vestibular ocular reflex that maintains balance whenever the body moves, and if there is an abnormality in the three semicircular canals, the otolith organs, the vestibular nerve that controls them, or the central structures associated with them, an appropriate vestibular ocular reflex do not occur, and thereby the body becomes difficult to maintain balance.

Therefore, in patients who complain of dizziness, specific eye movements are induced due to abnormalities in the vestibular ocular reflex, so in clinical practice, catching the abnormalities during vestibulo-ocular reflex are determined and used for diagnosis.

The eye has a three-dimensional form of movement: movement about two axes, horizontal and vertical, and rotational movement in relation to the vestibular reflex movement.

To date, commercially available measurements of eye movement have been performed by examination through the examiner's eyes, the Frenzel glasses, the electric nystagmography, the video nystagmography, or the video head impulse test.

However, in the method where the examiner directly observes the eye movement or uses the Frenzel glasses, when the amount of change in nystagmus is large, the eye movement may be observed, but in the opposite case, there is a problem in that measurement is difficult.

The video nystagmography has been widely used as a diagnostic method to date, where the user observes the eye movement through video and simultaneously displays three-axis measurements of the eye's horizontal, vertical, and rotational movements through graphs, which are then used for diagnosis.

However, the latter has an advantage of being able to measure objectively, but for 3-axis analysis, images acquired through a separate image sensor mounted inside are processed to measure the eye movements in 3 axes.

Therefore, sensitivity is determined by the resolution, frame rate, and image processing accuracy of the image sensor mounted inside, and because of the technical problem of having to separately measure the angular velocity measured along each axis of the eye movement, the problem is that it is difficult to use generally because it is expensive.

On the other hand, simple video Frenzel without these functions is relatively inexpensive and is widely used, but diagnosis using it has the limitation of requiring advanced training.

Meanwhile, the head impulse test is a part of a neurological examination that evaluates the vestibular ocular reflex. Video head-impulse test allows quantitative assessment of the bedside head-impulse test, and it is one of the main tests to determine central and peripheral dizziness and cerebellar dysfunction.

However, the conventional video head impulse test had limitations in that it had limited diagnostic value in clinical practice because it could only check the gain value of the head impulse test and whether the corrective saccades were made.

In particular, in central brain lesions, it was difficult to derive meaningful results other than making a diagnosis that the head impulse test showed normal findings.

However, recently, in addition to changes in gain of the head impulse test, cases showing abnormalities in the trajectory in central brain lesions have been reported both domestically and internationally, and have become a subject of interest.

However, until now, there has been no method of quantitatively evaluating such a vertical abnormal trajectory (perverted response), so there was a limitation in that medical staff had to visually check it to determine whether there was an abnormality.

### SUMMARY OF INVENTION

### Technical Problem

An object of the present invention is to provide an evaluation method for a head impulse test that may accurately evaluate results of a head impulse test by calculating a quantitative index when an abnormality in a vertical abnormality trajectory is shown in addition to a change in gain of a central brain lesion during the head impulse test.

An object of the present invention is not limited to the object mentioned above, and other objects and advantages of the present invention that are not mentioned may be understood from the following description and will be more clearly understood by embodiments of the present invention. In addition, it will be readily apparent that the objects and advantages of the present invention may be realized by means and combinations thereof as indicated in the claims.

### Technical Solution

An evaluation method for a head impulse test according to an embodiment of the present invention for achieving the object described above includes a step of extracting subject's eye and head movement data by a video nystagmograph; a step of detecting an eye movement that is not synchronized with a horizontal head movement by a head impulse tester; a step of obtaining corrective saccade data by removing noise from an unsynchronized eye movement signal detected in the head impulse tester by a filter unit; and a step of calculating a ratio of horizontal to vertical angular velocity of corrective saccades as a quantitative index by a control unit.

The evaluation method for a head impulse test according to an embodiment of the present invention may further include a step of diagnosing a central brain lesion using the calculated quantitative index by a diagnosis unit.

The step of acquiring the corrective saccade data may obtain corrective saccades that occur in addition to a vestibular ocular reflex during the head impulse test.

The step of acquiring the corrective saccade data may remove noise from a signal other than saccades using a low-pass filter by the filter unit.

An evaluation device for a head impulse test according to another embodiment of the present invention includes a video nystagmography that extracts subject's eye and head movement data; a head impulse tester that detects an eye movement that is not synchronized with a horizontal head movement; a filter unit that obtains corrective saccade data by removing noise from an unsynchronized eye movement signal detected in the head impulse tester; and a control unit that calculates a ratio of horizontal to vertical angular velocity of corrective saccades as a quantitative index.

The evaluation device for a head impulse test according to another embodiment of the present invention may further include a diagnosis unit that diagnoses a central brain lesion using the calculated quantitative index.

Details of other embodiments are included in “BEST MODE FOR INVENTION” and the accompanying “drawings.”

The advantages and/or features of the present invention and methods for achieving them will become clear by referring to the various embodiments described in detail below along with the accompanying drawings.

However, the present invention is not limited to the configuration of each embodiment disclosed below, but may also be implemented in various different forms, and each embodiment disclosed in this specification is intended to ensure that the disclosure of the present invention is complete, and is provided to fully inform those skilled in the art of the present invention of the scope of the present invention, and the present invention is only defined by each claim in the claims.

### Advantageous Effects

The present invention is dramatically helpful in the diagnosis of central brain lesions by detecting the vertical abnormal trajectory in the central brain lesions.

In addition, by calculating quantified data of the vertical abnormal trajectory, it is possible to determine treatment effectiveness and monitor disease progress, thereby improving the reliability and accuracy of disease diagnosis.

### BRIEF DESCRIPTION OF THE DRAWINGS

FIG. 1 is a block diagram of a device for implementing an evaluation method for a head impulse test according to an embodiment of the present invention.

FIG. 2 is a flowchart for explaining an operation of the evaluation method for a head impulse test according to an embodiment of the present invention.

FIG. 3 is a graph showing a horizontal head movement and an eye movement detected in step S200 in the evaluation method for a head impulse test shown in FIG. 2.

FIG. 4 is a graph showing a change in angular velocity of the head and eyes over time in a patient who shows a vertical trajectory abnormality according to horizontal head movements on both sides in step S400 in the evaluation method for a head impulse test shown in FIG. 2.

FIG. 5 is a graph showing a change in vertical eye angular velocity compared to the vertical head angular velocity of a patient who shows the vertical trajectory abnormality according to the horizontal head movements on both sides in step S400 in the evaluation method for a head impulse test shown in FIG. 2.

FIG. 6 is a graph showing a difference in gain of the vestibular ocular reflex in patients with brain lesions with damaged central vestibular organs diagnosed in step S500 in the evaluation method for a head impulse test shown in FIG. 2.

FIG. 7 is a diagram showing results of a head impulse test in a patient with multiple system atrophy who shows the vertical trajectory abnormality.

### BEST MODE FOR INVENTION

Hereinafter, preferred embodiments of the present invention will be described in detail with reference to the attached drawings.

Before explaining the present invention in detail, the terms or words used in this specification should not be construed as unconditionally limited to their ordinary or dictionary meanings, and the inventors of the present invention may use the concepts of various terms by being appropriately defined in order to explain the present invention in the best way.

Furthermore, it should be noted that these terms and words should be interpreted with meanings and concepts consistent with the technical idea of the present invention.

That is, the terms used in this specification are only used to describe preferred embodiments of the present invention, and are not used with the intention of specifically limiting the content of the present invention.

These terms are defined in consideration of various possibilities of the present invention.

In addition, in this specification, singular expressions may include plural expressions unless the context clearly indicates a different meaning.

In addition, it should be noted that even if similarly expressed in plural, it may have a singular meaning.

Throughout this specification, it may mean that when a component is described as “including” another component, it does not exclude any other component, but includes any other component, unless specifically stated to the contrary.

Furthermore, when a component is described as being “installed within or connected to” another component, this component may be installed by being connected to in direct or being in contact with the other component.

In addition, they may be installed at a certain distance, and in a case where they are installed at a certain distance, a third component or means may exist for fixing or connecting the component to another component.

Meanwhile, it should be noted that the description of the third component or means may be omitted.

On the other hand, in a case where a component is described as being “directly connected” or “directly coupled” to another component, it should be understood that no third component or means exists.

Likewise, it should be interpreted that other expressions that describe a relationship between each component, such as “between” and “immediately between”, or “neighboring” and “directly neighboring”, have the same meaning.

In addition, in this specification, terms such as “one surface”, “other surface”, “one side”, “other side”, “first”, “second”, etc. are used to clearly distinguish one component from another component.

However, it should be noted that the meaning of the corresponding component is not limited by such a term.

In addition, in this specification, it should be understood that terms related to positions such as “top”, “bottom”, “left”, “right”, etc., if used, indicate relative positions of the corresponding components in the drawings.

In addition, unless the absolute location is specified, it should be understood that these location-related terms do not refer to the absolute locations.

Moreover, in the specification of the present invention, terms such as “ . . . portion”, “ . . . unit”, “module”, “device”, etc., when used, mean units capable of processing one or more functions or operations.

It should be noted that this may be implemented by hardware or software, or a combination of hardware and software.

In the drawings attached to this specification, the size, position, connection relationship, etc. of each component constituting the present invention may be exaggerated, reduced, or omitted in order to transmit the idea of the present invention sufficiently clearly or for convenience of explanation, and therefore its proportions or scale may not be exact.

In addition, hereinafter, in describing the present invention, detailed descriptions of configurations that are determined to unnecessarily obscure the gist of the present invention, for example, known technologies including prior art, may be omitted.

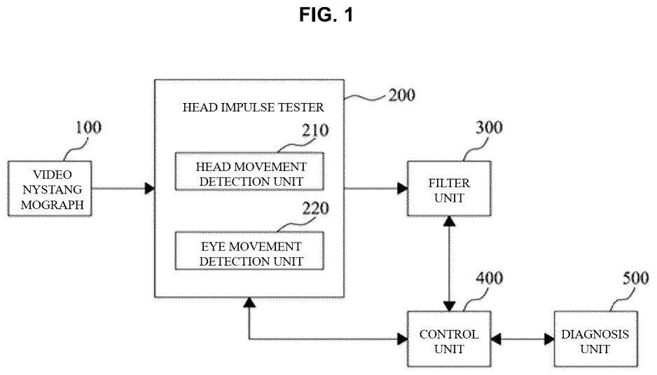

FIG. 1 is a block diagram of a device for implementing an evaluation method for a head impulse test according to an embodiment of the present invention, and includes a video nystagmograph 100, a head impulse tester 200, a filter unit 300, a control unit 400, and a diagnosis unit 500. The head impulse tester 200 includes a head movement detection unit 210 and an eye movement detection unit 220.

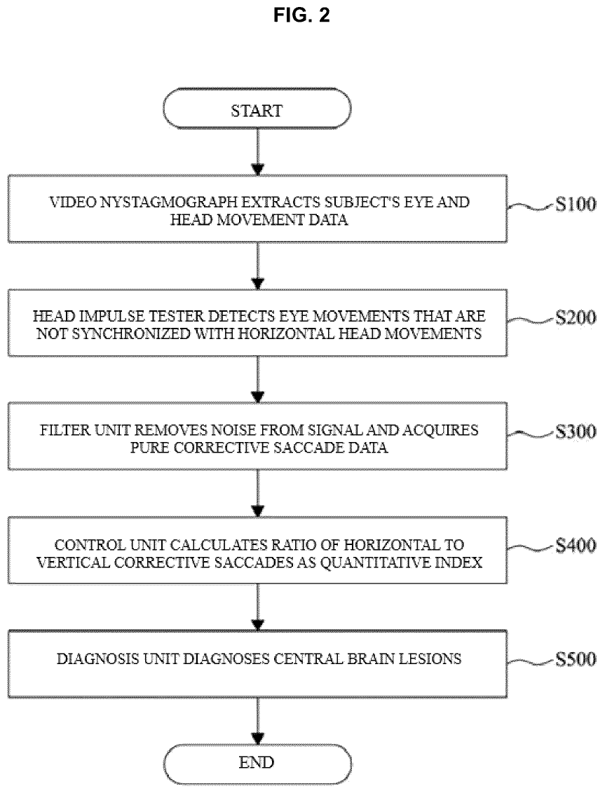

FIG. 2 is a flowchart for explaining an operation of the evaluation method for a head impulse test according to an embodiment of the present invention.

Referring to FIGS. 1 and 2, the operation of the evaluation method for a head impulse test according to an embodiment of the present invention will be briefly described as follows.

First, the video nystagmograph 100 extracts the subject's eye and head movement data (S100).

The head impulse tester 200 detects eye movements that are not synchronized with horizontal head movements (S200). That is, the head movement detection unit 210 detects the horizontal head movement, and the eye movement detection unit 220 detects the unsynchronized eye movement.

The filter unit 300 removes noise from an unsynchronized eye movement signal detected by the head impulse tester 200 and acquires corrective saccade data (S300).

The control unit 400 calculates a ratio of horizontal to vertical corrective saccades as a quantitative index (S400).

The diagnosis unit 500 diagnoses central brain lesions using the quantitative index calculated by the control unit 400 (S500).

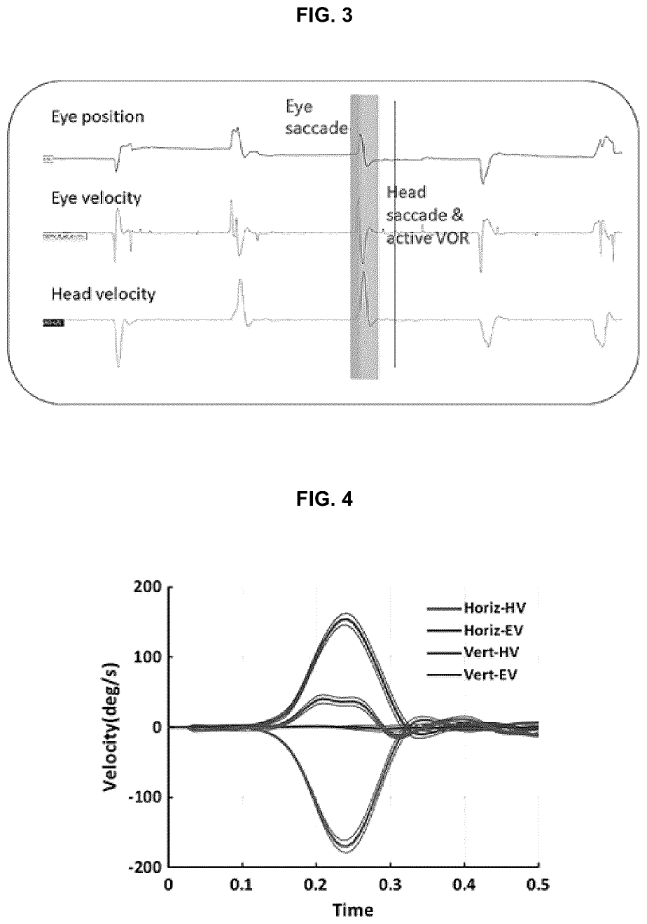

FIG. 3 is a graph showing the horizontal head movement and the eye movement detected in step S200 in the evaluation method for a head impulse test shown in FIG. 2.

FIG. 4 is a graph showing a change in angular velocity of the head and eyes over time in a patient who shows a vertical trajectory abnormality according to head movements on both sides in step S400 in the evaluation method for a head impulse test shown in FIG. 2.

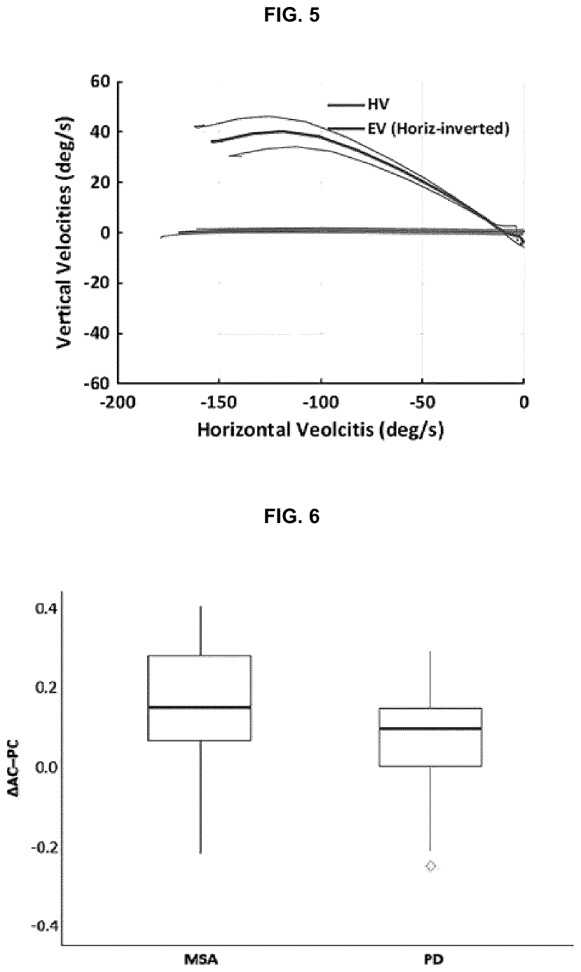

FIG. 5 is a graph showing a change in vertical eye angular velocity compared to the vertical head angular velocity of a patient who shows the vertical trajectory abnormality according to the head movements on both sides in step S400 in the evaluation method for a head impulse test shown in FIG. 2.

FIG. 6 is a graph showing a difference in gain of the vestibular ocular reflex in patients with brain lesions with damaged central vestibular organs diagnosed in step S500 in the evaluation method for a head impulse test shown in FIG. 2.

Referring to FIGS. 1 to 6, an organic operation of the evaluation method for a head impulse test according to an embodiment of the present invention will be described in detail as follows.

In general, the head impulse test is an essential test in the treatment of patients with dizziness and balance disorders. As shown in FIG. 3, in a state where the patient is looking at a fixation point in front of 1.2 m, the head impulse test checks abnormality of the vestibular ocular reflex that occur while rotating the head left and right and up and down at a rapid rate of 2 to 3 Hz.

In a case where the vestibular ocular reflex is normal, since the patient's immediate vestibular ocular reflex occurs due to rapid head rotation, the patient is able to continuously fixate on the fixation point in front, so no abnormality in eye or head movement is detected by the head impulse tester 200. On the other hand, in a case where the peripheral vestibular system and/or the central vestibular system (cerebellum and brain stem) involved in the vestibular ocular reflex are damaged, since the gain of the vestibular ocular reflex is reduced, catch-up saccades appear to compensate for this, so the head impulse tester 200 may detect the catch-up saccades.

By using this, the control unit 400 quantitatively calculates functions of the horizontal canal, the anterior canal, and the posterior canal, and the diagnosis unit 500 may evaluate using the calculated results.

Conventionally, until now, since the abnormality in the head impulse test could confirm only a decrease in the gain value and the presence of corrective saccades, in patients with central brain lesion disorders, the head impulse test was frequently found to be normal through conventional analysis methods, so its diagnostic value was limited.

Accordingly, in the present invention, the video nystagmograph 100 extracts the subject's eye movement and head movement data using Matlab software.

By using this, when the head impulse tester 200 performs the horizontal head impulse test, as shown in FIGS. 4 and 5, the head movement detection unit 210 and the eye movement detection unit 220 detect signals of separated eye movements that are not synchronized with the horizontal head movements.

The filter unit 300 removes noise from the signal for the unsynchronized eye movements detected by the head impulse tester 200 and obtains corrective saccade data.

The filter unit 300 uses a low pass filter to remove noise from signals other than the saccades.

The control unit 400 receives the corrective saccade data in which noise is removed from the filter unit 300, calculates the ratio of horizontal to vertical corrective saccades as the quantitative index, and determines vertically abnormal trajectory responses. In other words, the ratio of the horizontal to vertical angular velocity (deg/s) of the corrective saccades is calculated as the quantitative index.

The diagnosis unit 500 receives the quantitative index for the ratio of the calculated horizontal to vertical angular velocity of the patient's corrective saccades from the control unit 400 and compares it with data from normal people or patients with peripheral vestibulopathy to accurately diagnose the central brain lesions.

Through actual experimental results, as shown in FIG. 4, the patient who shows the vertical trajectory abnormality was confirmed to have a change in eye angular velocity according to the head movement on both sides over time, and as shown in FIG. 5, the changes in the vertical eye angular velocity compared to the vertical head angular velocity were confirmed.

In the graph shown in FIG. 4, Horiz-HV is the horizontal head velocity, Horiz-EV is the horizontal eye velocity, Vert-HV is the vertical head velocity, and Vert-EV is the vertical eye velocity. Referring to FIG. 4, during the horizontal head impulse test, the horizontal eye movement occurs in the opposite direction of the head movement. In addition, the head has only horizontal movement and almost no vertical movement, so the vertical head velocity appears close to 0. However, the abnormal vertical eye movement was detected, thereby showing the vertical trajectory abnormality.

In the graph shown in FIG. 5, HV is the head velocity and EV is the eye velocity. Referring to FIG. 5, in a patient who shows the vertical trajectory abnormality while performing the horizontal head impulse test, when comparing the vertical eye velocity to the vertical head velocity, the head moves only in the horizontal direction and does not move vertically, so the vertical head velocity is 0. On the other hand, the eyes are deflected upward, and the corrective saccades (for example, corrective saccades of vertical trajectory abnormalities) appear to compensate for this.

In addition, it was confirmed that differences in abnormal trajectory movements appeared in patients with central brain lesions (30 Parkinson's disease patients and 23 multiple system atrophy patients) diagnosed in the diagnosis unit 500, as shown in FIG. 6.

Furthermore, it was confirmed that in patients with damage to the central vestibular system (cerebellum and brain stem) (e.g., patients with multiple system atrophy), the vertical trajectory abnormality appears more frequently than in Parkinson's disease patients, and the vertical trajectory abnormality appears quantitatively largely. It was confirmed that a cause of the vertical trajectory abnormality is associated with a large difference in gain of the vestibular ocular reflex (ΔACs-PCs) between the anterior canals (AC) and the posterior canals (PC) is large. Based on that, the vertical trajectory catch-up saccades (i.e. perverted) occurs due to the difference in gain of the vertical semicircular canal generated by degeneration of the cerebellum and brain stem.

In addition to Parkinson's disease and multiple system atrophy shown in FIG. 6, the present invention may be applied to dizziness, headache, cerebral infarction, hereditary or acquired cerebellar ataxia, multiple sclerosis, and other cranial nervous system diseases affecting the cerebellum, brain stem, and vestibular nervous system.

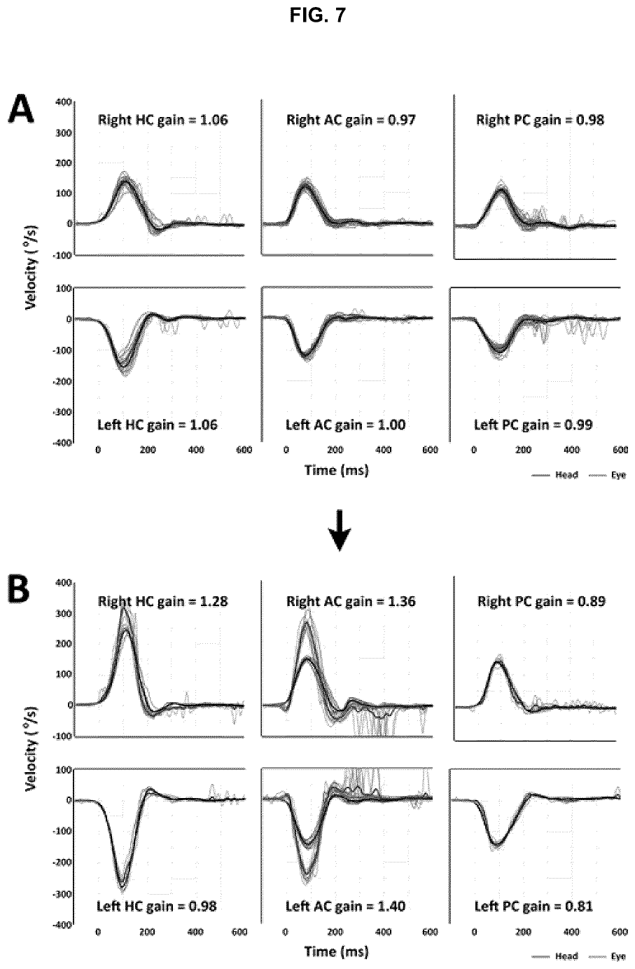

FIG. 7 is a diagram showing results of the head impulse test in a patient with multiple system atrophy who shows the vertical trajectory abnormality.

Referring to FIG. 7, it may be seen that the patient with early multiple system atrophy does not show the vertical trajectory abnormality during the head impulse test, and the difference in gain between the anterior and posterior semicircular canals is not significant. It may be seen that after 1 year, when the cerebellar degeneration progresses, patients with multiple system atrophy begin to develop the vertical trajectory abnormality, and the difference in gain between the anterior and posterior semicircular canals increases.

In addition, in patients with damage to the cerebellum and brain stem, in addition to the vertical trajectory abnormality, the eyes cannot focus on the fixation point during the head impulse test due to the increase in gain of the vestibular ocular reflex. To correct this, the diagnosis may be made by detecting the additional appearance of reversed catch-up saccades, in which the corrective saccades occur in the direction opposite to the direction of the head impulse test.

Meanwhile, in the case of the peripheral vestibular dysfunction disease (e.g., vestibular neuritis), the vertical trajectory abnormality described above does not occur, so the diagnosis unit 500 sets, as a reference value, a normal value of the vertical trajectory that is measured and calculated from a group of vestibular neuritis patients of the same age and gender, and may determine and diagnose central brain lesions based on this.

Meanwhile, predictions for regression analysis of patients with multiple system atrophy compared to Parkinson's disease according to an embodiment of the present invention are shown in [Table 1] below.

TABLE 1

| | Univariate analysis | | Multivariate analysis* | |
|---|---|---|---|---|
| Variables | OR (95% CI) | p value | OR (95% CI) | p value |
| Age, years | 0.99 (0.94-1.04) | 0.610 | | |
| Female sex | 0.31 (0.10-0.96) | 0.042 | 0.20 (0.02-1.62) | 0.131 |
| Reversed catch-up saccades | 10.24 (1.13-92.38) | 0.038 | 22.79 (1.09-478.20) | 0.044 |
| Perverted catch-up saccades | 12.69 (1.43-112.51) | 0.023 | 16.42 (1.15-235.09) | 0.039 |
| ΔACs − PCs | 187.48 (1.83-19,251.89) | 0.027 | 10.25 (0.01-17,280.37) | 0.539 |
| CPN | 22.31 (2.58-192.90) | 0.005 | 33.55 (2.99-376.55) | 0.004 |
| Downbeat nystagmus | 0.41 (0.04-4.21) | 0.453 | | |
| Horizontal GEN | 22.31 (2.58-192.90) | 0.005 | 18.75 (0.89-394.09) | 0.059 |
| Perverted HSN | 0.91 (0.25-3.36) | 0.891 | | |

It may be seen that the vertical abnormality perverted catch-up saccades value calculated using the evaluation device of the head impulse test of the present invention increases in direct proportion to the difference in gains between the anterior and the posterior semicircular canals. In particular, in the case of multiple system atrophy, an odds value of the calculated vertical abnormality (i.e. perverted meaning unwanted vertical bias during horizontal impulses or horizontal bias during vertical impulses) catch-up saccade value may increase by 16 times.

In other words, it may be seen that the vertical trajectory abnormality in the evaluation device of the head impulse test diagnoses multiple system atrophy more accurately and effectively than simply calculating the difference in gain between the anterior and posterior semicircular canals.

As such, the present invention provides the evaluation method for a head impulse test that may accurately evaluate the results of the head impulse test by calculating the quantitative index when an abnormality in the vertical abnormality trajectory is shown in addition to a change in gain of the central brain lesion during a horizontal head impulse test.

Through this, the present invention is dramatically helpful in the diagnosis of central brain lesions by detecting the vertical or horizontal abnormal trajectory during head impulse test in the central brain lesions.

In addition, by calculating quantified data of the vertical and horizontal abnormal trajectories, it is possible to determine treatment effectiveness and monitor disease progress, thereby improving the reliability and accuracy of disease diagnosis.

Although various preferred embodiments of the present invention have been described above by giving some examples, the description of the various embodiments described in the “BEST MODE FOR INVENTION” section is merely illustrative and those skilled in the art will understand from the above description that the present invention may be implemented with various modifications or equivalent implementations of the present invention.

In addition, it should be noted that since the present invention may be implemented in various other forms, the present invention is not limited by the above description, and the above description is intended to make the disclosure of the present invention complete and is only provided to fully inform those skilled in the art of the present invention of the scope of the present invention, and the present invention is only defined by each claim in the claims.

1. An evaluation method for a head impulse test comprising:
a step of extracting subject's eye and head movement data by a video nystagmograph;
a step of detecting an eye movement that is not synchronized with a horizontal head movement by a head impulse tester;
a step of obtaining corrective saccade data by removing noise from an unsynchronized eye movement signal detected in the head impulse tester by a filter unit; and
a step of calculating a ratio of horizontal to vertical angular velocity of corrective saccades as a quantitative index by a control unit.

2. The evaluation method for a head impulse test according to claim 1, further comprising:
a step of diagnosing a central brain lesion using the calculated quantitative index by a diagnosis unit.

3. The evaluation method for a head impulse test according to claim 1,
wherein in the step of acquiring the corrective saccade data,
obtaining corrective saccades that occur in addition to a vestibular ocular reflex during the head impulse test.

4. The evaluation method for a head impulse test according to claim 3,
wherein in the step of acquiring the corrective saccade data,
removing noise from a signal other than saccades using a low-pass filter by the filter unit.

5. An evaluation device for a head impulse test comprising:
a video nystagmograph that extracts subject's eye and head movement data;
a head impulse tester that detects an eye movement that is not synchronized with a horizontal head movement;
a filter unit that obtains corrective saccade data by removing noise from an unsynchronized eye movement signal detected in the head impulse tester; and
a control unit that calculates a ratio of horizontal to vertical angular velocity of corrective saccades as a quantitative index.

6. The evaluation device for a head impulse test according to claim 5, further comprising:
a diagnosis unit that diagnoses a central brain lesion using the calculated quantitative index.
